(19) United States
(12) Patent Application Publication
(10) Pub. No.: US 2006/0241526 A1
(43) Pub. Date: Oct. 26, 2006

(54) Therapeutic shockwave system with automatically controlled ramping

(76) Inventors: Markus Lanski, Nurnberg (DE); Christian Meinert, Marloffstein (DE)

Correspondence Address: SCHIFF HARDIN, LLP;PATENT DEPARTMENT, 6600 SEARS TOWER, CHICAGO, IL, 60606-6473, US

(21) Appl. No.: 11/366,232

(22) Filed: Mar. 1, 2006

(30) Foreign Application Priority Data
Mar. 1, 2005 (DE) 10 2005 009 904.1

(51) Int. Cl. A61H 1/00; A61B 8/00

(52) U.S. Cl. 601/2; 600/439

(57) ABSTRACT

A shockwave system has a shockwave source that generates shockwaves for a treatment of a patient. At the beginning of the treatment the shockwave energy, starting from a predeterminable energy start value, is automatically, successively increased in predeterminable energy levels to a predeterminable energy end value. Such a shockwave system enables an optimized beginning of a shockwave treatment.

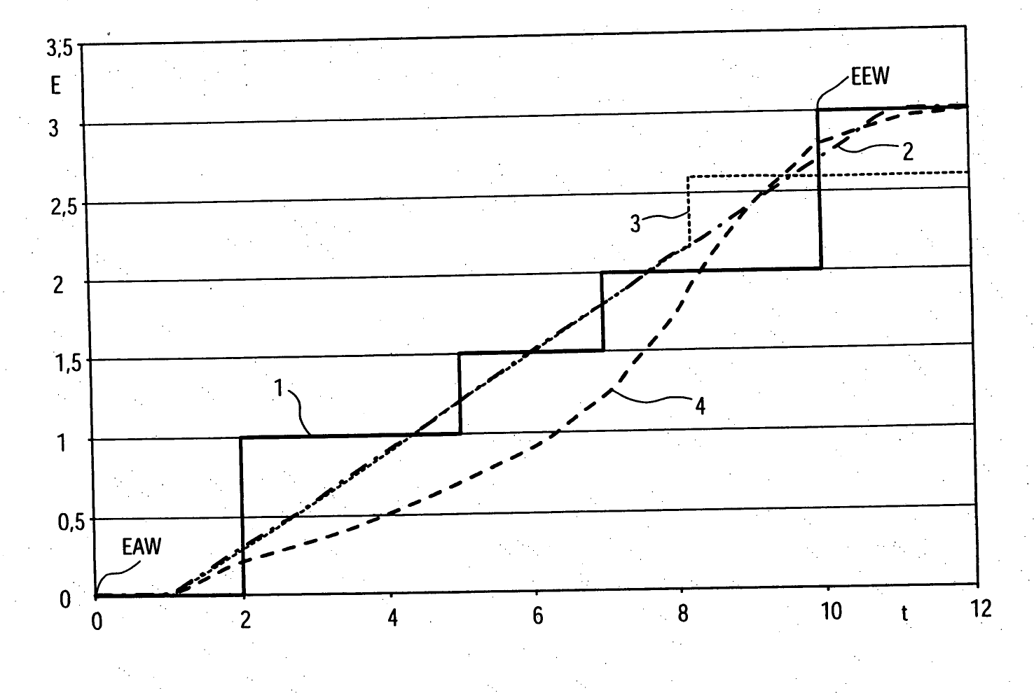

### BACKGROUND OF THE INVENTION

1. Field of the Invention

The invention concerns a shockwave system with a shockwave source that generates shockwaves for a treatment of a patient.

2. Description of the Prior Art

Shockwave systems of the above type serve for treatment of the patient with extra-corporeal shockwaves, in the majority of cases for lithotripsy or pain therapy, in particular in extra-corporeal shockwave therapy (ESWT).

Lithotripsy is a therapeutic method to destroy a calculus (for example a gallstone or kidney stone) located in the body of an organism without a surgical procedure, by using focused shockwaves. Both in lithotripsy and in extra-corporeal shockwave therapy, at the beginning the treatment is begun with low energy values in order to acclimate the patient to the treatment and the pain that may be associated with the treatment. This slow increase of the energy values that the operator manually implements is known as ramping. It is the goal to reach an optimally high energy level for the particular application in order to ensure an effective therapy.

### SUMMARY OF THE INVENTION

An object of the present invention is to provide a shockwave system of the type described above that enables an optimized beginning of a shockwave treatment.

This object is inventively achieved by a shockwave system having a shockwave source that generates shockwaves for treatment of a patient, wherein at the beginning of the treatment the shockwave energy, starting from a predeterminable energy initial value, is automatically, successively increased in predeterminable energy levels (stages) to a predeterminable energy end value.

The inventive shockwave system enables an optimized beginning of the shockwave treatment in patients since the energy levels can be calibrated significantly more finely than in the case of a manual increase. For this purpose, with each individual shockwave the high voltage that determines the energy value is increased by only a very small amount. In this manner, changes are possible that correspond to only a fraction of a conventional (manual) increase of the energy values. A very gentle (conservative) acclimation of the patient to the necessary energy end value is thereby possible since a spiking or jumping increase of the energy values, as well as a severe increase of the pain sensation that is associated therewith is reliably avoided.

Since the increase to the energy end value ensues automatically, only the parameters for the energy levels must be predetermined by the operator (this can, for example, ensue in the startup of the shockwave system), and the shockwave system can be started at a predeterminable energy start value. Generally, an intervention by the operator is not necessary until reaching the energy end value.

The increase of the shockwave energy can be parameterized in multiple ways in the context of the invention. For example, the predeterminable energy start value at which the treatment is begun can be constant, in particular zero.

According to a further embodiment, the energy start value is predeterminable dependent on the energy end value applied in the last treatment. In the event that a certain acclimation to the shockwaves has already been reached with a patient, a higher energy start value with than the normal minimal energy start value can be used as a starting point. The time until reaching the energy end value necessary for a successful therapy thus can be distinctly shortened.

In a further embodiment, the successive increase of the shockwave energy can be implemented in fine stages, such that the increase essentially exhibits a constant slope. The more finely staged that the increase of the shockwave energy is implemented; the more gentle the acclimation of the patient to the energy end value necessary for the application.

This slope (curve) of the increase of the shockwave energy can essentially form a straight line, but other curves deviating from a straight line are also possible for the increase of the shockwave energy.

The curve of the increase of the shockwave energy can be defined by a predeterminable function. This function can be predetermined, for example, dependent on the energy end value applied in the treatment.

In another embodiment of the shockwave system according to the invention, the automatic increase of the shockwave energy automatically terminates given a manual intervention, and the system automatically switches into a manual mode.

Furthermore, in the context of the invention the shockwave energy can be manually adjusted (set) to a new energy end value after reaching the energy end value applied in the last treatment.

In particularly user-friendly embodiments of the inventive shockwave system, values and/or the intermediate values of the energy levels can be displayed, or the curve of the increase of the shockwave energy, and/or curve of the intermediate values, can be graphically represented.

Through the aforementioned parameterization possibilities, the curve of the increase of the shockwave energy can be adapted in a simple manner to different treatment methods and to the different pain sensitivities of individual patients. User-specific application variants can be achieved in a simple manner with this approach. This parameterization can ensue either once by a service setting at the first startup, or during a maintenance (service) visit, or by the operator.

### DESCRIPTION OF THE DRAWING

The single FIGURE is a graph of energy versus time, illustrating the automatic ramping of the energy of a shockwave source in accordance with the present invention.

### DESCRIPTION OF THE PREFERRED EMBODIMENTS

The automatic ramping of the energy of a shockwave source in accordance with the invention can be embodied in the high voltage supply and/or a control unit therefor, for an extracorporeal shockwave generator of the type describe in U.S. Pat. No. 4,905,675 or U.S. Pat. No. 4,674,505. The teachings of both of those patents are incorporated herein by reference.

In the drawing, time t is plotted as a dimensionless quantity on the abscissa and the shockwave energy E is plotted as a dimensionless quantity on the ordinate.

As exemplary curve for a manual increase of the shockwave energy E is designated 1. Three examples of curves for an automatic increase of the shockwave energy E, which curves are achievable with the inventive shockwave system, are designated 2, 3 and 4.

The treatment shown in the drawing is manually begun at an energy start value EAW=0 at t=0, whereby the energy levels exhibit different levels as well as different amplitudes.

The shockwave energy is increased to a value E=1 at the point in time t=2. The shockwave energy is increased to E=1.5 at t=5 and to 2 at t=7. At t=10, the shockwave energy is raised to its energy end value EEW=3.

Relative to the manual increase of the shockwave energy, the automatic increase of the shockwave energy in the inventive shockwave system ensues successively and in fine stages, such that the increase essentially exhibits a constant slope 2. In the shown exemplary embodiment, the slope 2 of the increase of the shockwave energy E essentially forms a straight line.

The selected curve 2 begins at t=0 with an energy start value that lies at EAW=0 until the point in time t=1 and then, for example, exhibits a slope of 0.3. The energy end value EEW=3 is, for example, reached at t=11.5.

From the comparison of both curves 1 and 2, it is apparent that an increase of the shockwave energy according to the curve 2 is gentler for the patient than an increase according to curve 1.

The curve 3 of the shockwave energy E differs from the curve 2 in that, at the point in time t=8.1 and at a shockwave energy of E=2.1, the user effects a manual increase to an energy end value EEW 2.6.

The curve 4 of the shockwave energy E, which deviates from the curves 2 and 3, is in particular advantageous given patients with a high sensitivity to pain, for example children and older people.

The curve 4 begins with an energy start value that lies at EAW=0 until the point in time t=1 and then continuously remains below the shockwave energy according to the curve 2 until a shockwave energy of E=2.4 at the point in time t=9. The energy end value EEW=3 is only reached at t=12, and therewith later than in the curve 2 of the shockwave energy E.

From the explanation of the (non-limiting) exemplary embodiments of the invention, it is clear that an optimized beginning of a shockwave treatment is possible in a simple manner with the inventive shockwave system.

Although modifications and changes may be suggested by those skilled in the art, it is the intention of the inventors to embody within the patent warranted hereon all changes and modifications as reasonably and properly come within the scope of their contribution to the art.

We claim as our invention:

1. A shockwave system comprising:
a shockwave source that generates shockwaves, having an energy content, for treatment of a patient; and
a control unit that operates said shockwave source to start, at a beginning of said treatment, from a predetermined energy for the shockwaves, and then automatically successively increases said energy in predetermined energy steps to a predetermined energy end value.

2. A shockwave system as claimed in claim 1 wherein said control unit sets said energy start value at a constant value.

3. A shockwave system as claimed in claim 2 wherein said control unit sets said energy start value at zero.

4. A shockwave system as claimed in claim 1 wherein said control unit sets said energy start value dependent on said energy end value applied in an immediately preceding treatment.

5. A shockwave system as claimed in claim 1 wherein said control unit successively increases said energy in fine steps so that an increase in said energy exhibits a substantially constant step.

6. A shockwave system as claimed in claim 5 wherein said control unit sets a curve for said increase that substantially forms a straight line.

7. A shockwave system as claimed in claim 5 wherein said control unit sets a curve for the increase of said energy according to a predetermined function.

8. A shockwave system as claimed in claim 6 wherein said control unit predetermines said function dependent on an energy end value applied in an immediately preceding treatment.

9. A shockwave system as claimed in claim 1 wherein said control unit automatically deactivates said automatic increase of said energy upon a manual intervention, and then switches into a manual mode for operating said shockwave source.

10. A shockwave system as claimed in claim 1 wherein said control unit allows manual adjustment of said energy to a new energy end value after reaching said energy and value applied in a preceding treatment.

11. A shockwave system as claimed in claim 1 wherein said control unit comprises a display at which at least one of said energy start value, said energy end value and values therebetween are displayed.

12. A shockwave system as claimed in claim 1 wherein said control unit comprises a display at which a curve of the increase of said energy is graphically displayed.
